(19) United States
(12) Patent Application Publication
(10) Pub. No.: US 2007/0161837 A1
(43) Pub. Date: Jul. 12, 2007

(54) Zeolitic transalkylation with aluminum chloride alkylation

(75) Inventors: James R. Butler, League City, TX (US); Jean Bernard Cary, LeHavre (FR)

Correspondence Address: David J. Alexander, P.O. Box 674412, Houston, TX, 77167, US

(73) Assignee: Fina Technology, Inc., Houston, TX (US)

(21) Appl. No.: 11/326,659

(22) Filed: Jan. 7, 2006

(51) Int. Cl. C07C 15/067; C07C 2/64; B01J 8/04

(52) U.S. Cl. 585/446; 422/190

(57) ABSTRACT

Alkylation processes are described herein. The alkylation process generally includes contacting a first aromatic compound with an alkylation catalyst including a metal halide catalyst in the presence of an alkylating agent to form an alkylation output stream including a second aromatic compound and contacting at least a portion of the output stream with a transalkylation catalyst including a zeolite catalyst in the presence of the first aromatic compound to form a transalkylation output stream including the second aromatic compound.

### FIELD

Embodiments of the present invention generally relate to alkylation systems including aluminum chloride catalyst based alkylation in combination with zeolitic catalyst based transalkylation.

### BACKGROUND

As reflected in the patent literature, many alkylation/transalkylation systems utilize Friedel-Crafts type catalysts, such as aluminum chloride. Such systems generally result in high catalyst activities at low reaction temperatures and feed molar ratios.

However, Friedel-Crafts systems result in highly corrosive by-products which are present in the alkylation output. Accordingly, high investment and maintenance costs are incurred to ensure the numerous parts of the plant are corrosion resistant. Further, the Friedel-crafts catalyst, which is dissolved or suspended in the reaction mixture, must be removed from the product stream and disposed of, which may result in aluminum-containing waste requiring disposal in compliance with environmental regulations.

While non-corrosive catalyst systems, such as zeolite catalyst systems, have been used for alkylation processes, such zeolite systems are generally grassroots plants, requiring significant capital investment. Further, such catalyst are generally of lower activity, generally requiring higher reaction temperatures and feed molar ratios, which still may result in lower output yields. Further, such high temperatures and feed ratios generally deactivate the catalyst at a high rate, resulting in frequent catalyst replacement/regeneration.

Therefore, a need exists to develop an alkylation/transalkylation system utilizing existing Friedel-Crafts alkylation systems, while minimizing waste and system maintenance and retaining high system yields.

### SUMMARY

Embodiments of the present invention include an alkylation process. The alkylation process generally includes contacting a first aromatic compound with an alkylation catalyst including a metal halide catalyst in the presence of an alkylating agent to form an alkylation output stream including a second aromatic compound and contacting at least a portion of the output stream with a transalkylation catalyst including a zeolite catalyst in the presence of the first aromatic compound to form a transalkylation output stream including the second aromatic compound.

Embodiments of the invention further include an alkylation system including an alkylation reaction zone including a metal halide alkylation catalyst and a transalkylation reaction zone, wherein the alkylation system is adapted to consume about at least 50 percent less metal than an alkylation system wherein both the alkylation reaction zone and the transalkylation zone include a metal halide alkylation catalyst.

Embodiments of the invention further include a method of retrofitting an alkylation system. The method generally includes providing an alkylation/transalkylation system adapted for aluminum chloride based alkylation, wherein the alkylation/transalkylation system includes an alkylation reaction zone in operable communication with a first transalkylation reaction zone, disconnecting the first transalkylation zone from the alkylation reaction zone and placing a second transalkylation zone in operable communication with the alkylation reaction zone, wherein the second transalkylation zone comprises a zeolite based alkylation catalyst.

### BRIEF DESCRIPTION OF DRAWINGS

FIGS. 1A and 1B illustrate an embodiment of an alkylation/transalkylation process.

### DETAILED DESCRIPTION

Introduction and Definitions

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions when the information in this patent is combined with available information and technology.

Various terms as used herein are shown below. To the extent a term used in a claim is not defined below, it should be given the broadest definition persons in the pertinent art have given that term as reflected in printed publications and issued patents. Further, unless otherwise specified, all compounds described herein may be substituted or unsubstituted and the listing of compounds includes derivatives thereof.

The term “activity” refers to the weight of product produced per weight of the catalyst used in a process per hour of reaction at a standard set of conditions (e.g., grams product/gram catalyst/hr).

The term “alkylation” refers to the addition of an alkyl group to another molecule.

The term “deactivated catalyst” refers to a catalyst that has lost enough catalyst activity to no longer be efficient in a specified process. Such efficiency is determined by individual process parameters.

The term “processing” is not limiting and includes agitating, mixing, milling, blending and combinations thereof, all of which are used interchangeably herein. Unless otherwise stated, the processing may occur in one or more vessels, such vessels being known to one skilled in the art.

The term “recycle” refers to returning an output of a system as input to either that same system or another system within a process. The output may be recycled to the system in any manner known to one skilled in the art, for example, by combining the output with an input stream or by directly feeding the output into the system. In addition, multiple input/recycle streams may be fed to a system in any manner known to one skilled in the art.

The term “regenerated catalyst” refers to a catalyst that has regained enough activity to be efficient in a specified process. Such efficiency is determined by individual process parameters.

The term “regeneration” refers to a process for renewing catalyst activity and/or making a catalyst reusable after its activity has reached an unacceptable/inefficient level. Examples of such regeneration may include passing steam over a catalyst bed or burning off carbon residue, for example.

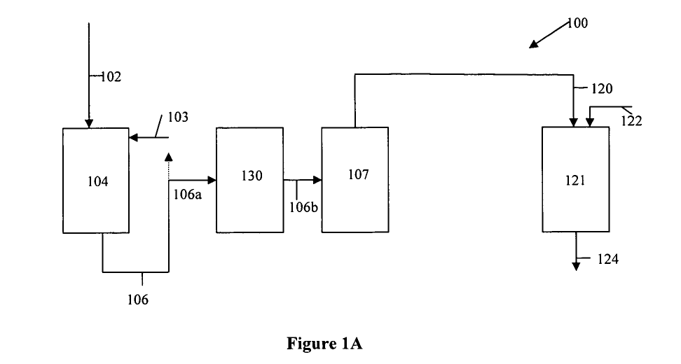

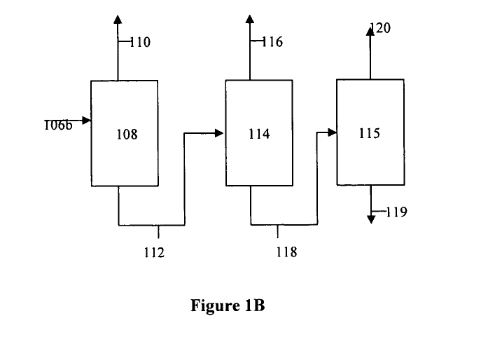

FIG. 1B illustrates a schematic block diagram of an embodiment of an alkylation/transalkylation process 100. Although not shown herein, the process stream flow may be modified based on unit optimization so long as the modification complies with the spirit of the invention, as defined by the claims. For example, at least a portion of any overhead fraction may be recycled as input to any other system within the process and/or any process stream may be split into multiple process stream inputs, for example. Also, additional process equipment, such as heat exchangers, may be employed throughout the processes described herein and such placement is generally known to one skilled in the art. Further, while described below in terms of primary components, the streams indicated below may include any additional components as known to one skilled in the art.

The process 100 generally includes supplying an input stream 102 to an alkylation system 104. The alkylation system 104 is generally adapted to contact the input stream 102 with an alkylation catalyst to form an alkylation output stream 106. In addition to the input stream 102, an additional input, such as an alkylating agent, is generally supplied to the alkylation system 104 via line 103.

At least a portion of the alkylation output stream 106 passes to a separation system 107. The separation system 107 generally includes a plurality of vessels, such vessels being adapted to separate components of the output stream 106b. At least a portion of the separation system output, described in further detail below, is passed from the separation system 107 to a second alkylation system (i.e., a transalkylation system 121) as transalkylation input 120.

In addition to the transalkylation input 120, an additional input, such as additional aromatic compound, is generally supplied to the transalkylation system 121 via line 122 to contact a transalkylation catalyst and form a transalkylation output 124.

The input stream 102 generally includes a first aromatic compound. The first aromatic compound may include substituted or unsubstituted aromatic compounds. If present, the substituents on the aromatic compounds may be independently selected from alkyl, aryl, alkaryl, alkoxy, aryloxy, cycloalkyl, halide and/or other groups that do not interfere with the alkylation reaction, for example. Examples of substituted aromatic compounds generally include toluene, xylene, isopropylbenzene, normal propylbenzene, alpha-methylnaphthalene, ethylbenzene, mesitylene, durene, cymene, butylbenzene, pseudocumene, o-diethylbenzene, m-diethylbenzene, p-diethylbenzene, isoamylbenzene, isohexylbenzene, pentaethylbenzene, pentamethylbenzene, 1,2,3,4-tetraethylbenzene, 1,2,3,5-tetramethylbenzene, 1,2,4-triethylbenzene, 1,2,3-trimethylbenzene, m-butyltoluene, p-butyltoluene, 3,5-diethyltoluene, o-ethyltoluene, p-ethyltoluene, m-propyltoluene, 4-ethyl-m-xylene, dimethylnaphthalenes, ethylnaphthalene, 2,3-dimethylanthracene, 9-ethylanthracene, 2-methylanthracene, o-methylanthracene, 9,10-dimethylphenanthrene and 3-methyl-phenanthrene. Further examples of first aromatic compounds include hexylbenzene, nonylbenzene, dodecylbenzene, pentadecylbenzene, hexyltoluene, nonyltoluene, dodecyltoluene and pentadecytoluene. In another embodiment, the first aromatic compound includes one or more hydrocarbons, such as benzene, naphthalene, anthracene, naphthacene, perylene, coronene and phenanthrene, for example. In another embodiment, the first aromatic compound includes benzene.

The benzene may be supplied from a variety of sources, such as a fresh benzene source and/or a variety of recycle sources. As used herein, the term “fresh benzene source” refers to a source including at least about 95 wt. % benzene, at least about 98 wt. % benzene or at least about 99 wt. % benzene, for example.

The alkylating agent 103 may include olefins (e.g., ethylene, propylene, butene and pentene), alcohols (e.g., methanol, ethanol, propanol, butanol and pentanol), aldehydes (e.g., formaldehyde, acetaldehyde, propionaldehyde, butyraldehyde and n-valeraldehyde) and/or alkyl halides (e.g., methyl chloride, ethyl chloride, propyl chloride, butyl chloride and pentyl chloride), for example. Additional alkylating agents known in the art may be utilized. In one embodiment, the alkylating agent includes a mixture of light olefins, such as mixtures of ethylene, propylene, butene and/or pentenes, for example. In another embodiment, the alkylating agent includes ethylene.

In addition to the first aromatic compound and the alkylating agent, the input stream 102 may further include other compounds in minor amounts (e.g., sometimes referred to as poisons or inactive compounds,) such as C₇ aliphatic compounds and/or nonaromatic compounds, for example. In one embodiment, the input stream 102 includes less than about 3% of such compounds or less than about 1%, for example.

The alkylation system 104 generally includes one or more reaction vessels, which are generally adapted to be corrosion resistant. Such reaction vessels may be liquid phase, vapor phase, supercritical phase or mixed phase reactors operated at temperatures and pressures sufficient to maintain the alkylation reaction in the corresponding phase, i.e., the phase of the aromatic compound, for example. Such temperatures and pressures are generally determined by individual process parameters. However, the alkylation reaction temperature may be from about 100° F. to about 500° F. and the pressure may be from about 30 psig to about 300 psig, for example.

The alkylation catalyst generally includes a Friedel-Crafts catalyst. Such Friedel-Crafts catalyst may include a metal halide, such as aluminum chloride. Alternatively, the alkylation catalyst may include sulfuric acid, phosphoric acid or hydrofluoric acid, for example. In a specific embodiment, the alkylation catalyst is aluminum chloride.

The alkylation process may further include cofeeding a catalyst promoter to the alkylation system 104. The catalyst promoter may include a hydrogen halide, such as hydrogen chloride or hydrogen bromide, or an alkyl halide, such as ethylene chloride and/or water, for example. In one embodiment, the catalyst promoter is fed to the alkylation system at a rate of about 40% to about 100% based on the weight of the alkylation catalyst. In one embodiment, the residence time of the alkylation system is from about 4 to about 30 minutes, for example.

The alkylation output 106 generally includes a second aromatic compound formed from the reaction of the first aromatic compound and the alkylating agent in the presence of the alkylation catalyst, for example.

The alkylation/transalkylation system 100 further includes a catalyst disposal stage 130. The catalyst disposal stage 130 is generally adapted to remove the alkylation catalyst from the output stream 106a and neutralize the same. For example, the removal may be effected by neutralization via series of aqueous and caustic washes. The removed catalyst may be recovered or sent for waste removal, for example. At least a portion of the neutralized alkylation output 106b is sent to the separation system 107.

The transalkylation system 121 generally includes one or more reaction vessels having a transalkylation catalyst disposed therein. The reaction vessels may include any reaction vessel, combination of reaction vessels and/or number of reaction vessels (either in parallel or in series) known to one skilled in the art.

In one embodiment, the transalkylation system 121 is operated under liquid phase conditions. For example, the transalkylation system 121 may be operated at a temperature of from about 150° F. to about 555° F. and a pressure of about 700 psig or less. In another embodiment, the transalkylation system 121 is operated under vapor phase conditions, for example. For example, the transalkylation system 121 may be operated at a temperature of from about 750° F. to about 850° F. and a pressure of from about 200 psig to about 400 psig.

The transalkylation catalyst may include a molecular sieve catalyst. Such molecular sieve catalyst may include zeolite beta, zeolite Y, zeolite MCM-22, zeolite MCM-36, zeolite MCM-49 or zeolite MCM-56, for example. In one embodiment, the catalyst is a zeolite beta having a silica to alumina molar ratio (expressed as SiO₂/Al₂O₃) of from about 10 to about 200 or about 20 to about 50, for example. In one embodiment, the zeolite beta may have a low sodium content, e.g., less than about 0.2 wt. % expressed as Na₂O, or less than about 0.02 wt. %, for example. The sodium content may be reduced by any method known to one skilled in the art, such as through ion exchange, for example. (See, U.S. Pat. No. 3,308,069 and U.S. Pat. No. 4,642,226 (formation of zeolite beta), U.S. Pat. No. 4,185,040 (formation of zeolite Y), U.S. Pat. No. 4,992,606 (formation of MCM-22), U.S. Pat. No. 5,258,565 (formation of MCM-36), WO 94/29245 (formation of MCM-49) and U.S. Pat. No. 5,453,554 (formation of MCM-56, which are incorporated by reference herein.))

The transalkylation catalyst may optionally be bound to, supported on or extruded with any support material. For example, the transalkylation catalyst may be bound to a support to increase the catalyst strength and attrition resistance. The support material may include alumina, silica, aluminosilicate, titanium and/or clay, for example.

The transalkylation output 124 generally includes the second aromatic compound, for example. As stated previously, any of the process streams, such as the transalkylation output 124 may be used for any suitable purpose or recycled back as input to another portion of the system 100, such as the separation system 107, for example.

In a specific embodiment, the first aromatic compound includes benzene and the alkylating agent includes ethylene. In one embodiment, the molar ratio of benzene to ethylene in the alkylation input stream 102 may be from about 1:1 to about 30:1, or from about 1:1 to about 20:1 or from about 1.5:1 to about 2.5:1, for example and the ratio of aluminum chloride to ethylene may be from about 0.001:1 to about 0.00025:1, for example. In one embodiment, the molar ratio of benzene to ethylene in the transalkylation input stream 120 may be from about 1:1 to about 30:1 or from about 5:1 to about 20:1, for example and the space velocity may be from about 2 to about 30, for example.

In a specific embodiment, the separation system (or product recovery) 107 includes three separation zones (illustrated in FIG. 1B) operated at conditions known to one skilled in the art. The first separation zone 108 may include any process or combination of processes known to one skilled in the art for the separation of aromatic compounds. For example, the first separation zone 108 may include one or more distillation columns, either in series or in parallel. The number of such columns may depend on the volume of the alkylation output 106 passing therethrough, for example. While the temperature and pressure are system specific, the first separation zone temperature may be from about 130° C. to about 170° C. and the pressure may be atmospheric pressure, for example.

The overhead fraction 110 from the first column 108 generally includes the first aromatic compound, such as benzene, for example.

The bottoms fraction 112 from the first separation zone 108 generally includes the second aromatic compound, such as ethylbenzene, for example. The bottoms fraction 112 further includes additional components, which may undergo further separation in the second separation zone 114 and third separation zone 115, discussed further below.

The second separation zone 114 may include any process known to one skilled in the art, for example, one or more distillation columns, either in series or in parallel. While the temperature and pressure are system specific, the second separation zone temperature may be from about 120° C. to about 240° C. and the pressure may be from about 100 kPa to about 300 kPa, for example.

The overhead fraction 116 from the second separation zone 114 generally includes the second aromatic compound, such as ethylbenzene, which may be recovered and used for any suitable purpose, such as the production of styrene, for example.

The bottoms fraction 118 from the second separation zone 114 generally includes heavier aromatic compounds, such as polyethylbenzene, cumene and/or butylbenzene, for example, which may undergo further separation in the third separation zone 115.

The third separation zone 115 generally includes any process known to one skilled in the art, for example, one or more distillation columns (not shown), either in series or in parallel. While the temperature and pressure are system specific, the second separation zone temperature may be from about 120° C. to about 340° C. and the pressure may be vacuum pressure, for example.

In a specific embodiment, the overhead fraction 120 from the third separation zone 115 may include diethylbenzene and liquid phase triethylbenzene, for example. The bottoms fraction 119 (e.g., heavies) may be recovered from the third separation zone 115 for further processing and recovery.

Unfortunately, the transalkylation catalyst generally experiences deactivation upon exposure to reaction. The life of the catalyst generally depends on process conditions and catalyst type. However, when regeneration of any catalyst within the system is desired, the regeneration procedure generally includes processing the deactivated catalyst at high temperatures, although the regeneration may include any regeneration procedure known to one skilled in the art.

Once a reactor is taken off-line, the catalyst disposed therein may be purged. Off-stream reactor purging may be performed by contacting the catalyst in the off-line reactor with a purging stream, which may include any suitable inert gas (e.g., nitrogen), for example. The off-stream reactor purging conditions are generally determined by individual process parameters and are generally known to one skilled in the art.

The catalyst may then undergo regeneration. The regeneration conditions may be any conditions that are effective for at least partially reactivating the catalyst and are generally known to one skilled in the art. For example, regeneration may include heating the catalyst to a temperature or a series of temperatures, such as a regeneration temperature of from about 50° C. to about 400° C. above the purging or reaction temperature, for example.

In one specific non-limiting embodiment, the alkylation catalyst is heated to a first temperature (e.g., 700° F.) with a gas containing nitrogen and about 2% oxygen, for example, for a time sufficient to provide an output stream having an oxygen content of about 0.5%. The catalyst may then be heated to a second temperature for a time sufficient to provide an output stream having an oxygen content of about 2.0%. The second temperature may be about 50° F. greater than the first temperature, for example. The second temperature is generally about 950° F. or less, for example. The catalyst may further be held at the second temperature for a period of time, or at a third temperature that is greater than the second temperature, for example.

Upon catalyst regeneration, the catalyst may then be reused for transalkylation, for example.

### EXAMPLE

Benzene (4.335 ml./min.) and ethylene (0.776 ml./min.) were fed to an alkylation reactor (RX 8 CSTR) having a volume of 300 ml at a temperature of 130° C. and a pressure of 160 psig. In addition, an fresh alkylation catalyst (28% AlCl₃) was fed to the reactor at a rate of 0.00735 g./min while a recycle alkylation catalyst was fed to the reactor at a rate of 0.05 g./min. The reaction mixture was agitated at 1500 rpm. The effluent contained 33.0088% ethylbenzene and 0.6899% non aromatics from GC analysis.

Unexpectedly, replacing the Friedel-Crafts alkylation system (e.g., retrofitting aluminum chloride systems) with a zeolite system results in a reduction of metal halide consumption of at least 80%. Further, retaining the Friedel-Crafts alkylation system allows the process to retain the high product yield attained with Friedel-Crafts catalyst, while minimizing the metal byproducts needing treatment.

While the foregoing is directed to embodiments of the present invention, other and further embodiments of the invention may be devised without departing from the basic scope thereof and the scope thereof is determined by the claims that follow.

What is claimed is:

1. An alkylation process comprising:
contacting a first aromatic compound with an alkylation catalyst comprising a metal halide catalyst in the presence of an alkylating agent to form an alkylation output stream comprising a second aromatic compound; and
contacting at least a portion of the output stream with a transalkylation catalyst comprising a zeolite catalyst in the presence of the first aromatic compound to form a transalkylation output stream comprising the second aromatic compound.

2. The process of claim 1, wherein the first aromatic compound comprises benzene, the alkylating agent comprises ethylene and the second aromatic compound comprises ethylbenzene.

3. The process of claim 1, wherein the metal halide catalyst comprises aluminum chloride.

4. The process of claim 1 further comprising passing the alkylation output stream to a neutralization system.

5. The process of claim 4, wherein the neutralization system comprises washing the metal halide catalyst.

6. The process of claim 1, wherein the zeolite catalyst comprises a zeolite Y or zeolite beta catalyst.

7. An alkylation system comprising:
an alkylation reaction zone comprising a metal halide alkylation catalyst and a transalkylation reaction zone, wherein the alkylation system is adapted to consume about at least 50 percent less metal than an alkylation system wherein both the alkylation reaction zone and the transalkylation zone comprise a metal halide alkylation catalyst.

8. The system of claim 7, wherein the alkylation system consumes at least 80 percent less metal than an alkylation system wherein both the alkylation reaction zone and the transalkylation reaction zone comprise a metal halide alkylation catalyst.

9. The system of claim 7, wherein the alkylation system is adapted to produce an ethylbenzene product yield of at least 30 percent.

10. The system of claim 7, wherein the alkylation system is adapted to produce an ethylbenzene product yield of at least 50 percent out of the alkylation reaction zone.

11. The system of claim 7, wherein the metal halide catalyst comprises aluminum chloride.

12. The system of claim 7, wherein the transalkylation reaction zone comprises a zeolite catalyst.

13. A method of retrofitting an alkylation system comprising:
providing an alkylation/transalkylation system adapted for aluminum chloride based alkylation, wherein the alkylation/transalkylation system comprises an alkylation reaction zone in operable communication with a first transalkylation reaction zone;
isolating the first transalkylation zone from the alkylation reaction zone; and
placing a second transalkylation zone in operable communication with the alkylation reaction zone, wherein the second transalkylation zone comprises a zeolite based alkylation catalyst.

14. The method of claim 13, wherein the alkylation reaction zone is in operable communication with a catalyst disposal system.

15. The method of claim 14, wherein the catalyst disposal system is in operable communication with the second transalkylation zone.

16. The method of claim 13, wherein the zeolite based alkylation catalyst comprises zeolite beta.
